(19) United States
(12) Patent Application Publication
(10) Pub. No.: US 2014/0058027 A1
(43) Pub. Date: Feb. 27, 2014

(54) Anti-Static Agent For Polymer Resins

(71) Applicant: PCC Chemax, Inc., Piedmont, SC (US)

(72) Inventors: Andrew Carol George, Greenville, SC (US); Adam Keith Henderson, Simpsonville, SC (US)

(73) Assignee: PCC Chemax, Inc., Piedmont, SC (US)

(21) Appl. No.: 14/010,994

(22) Filed: Aug. 27, 2013

Related U.S. Application Data
(60) Provisional application No. 61/693,567, filed on Aug. 27, 2012.

(51) Int. Cl. C08K 5/20; C07C 237/06

(52) U.S. Cl. CPC C08K 5/20; C07C 237/06
USPC 524/217; 564/224

(57) ABSTRACT

An anti-static agent for polymers is disclosed. The anti-stat agent may be an amidoamine. The amidoamine may also be blended with a polyglycerol ester.

### RELATED APPLICATIONS

The present application claims priority and is based upon U.S. Provisional Patent Application Ser. No. 61/693,567, filed on Aug. 27, 2012, which is incorporated herein by reference.

### BACKGROUND

Polymeric materials comprising either natural and/or synthetic polymers can accumulate a static charge on the surface of the polymer. Also, products made from these polymeric materials tend to accumulate and can retain a static charge for an extended period of time.

Static charge is typically generated through frictional contact between two surfaces. The frictional contact causes one surface to lose electrons, becoming positively charged. The other surface gains electrons, becoming negatively charged. The charged surfaces are said to have a build-up of static charge.

Accumulation of a static charge can create many problems in both the manufacture and application of the polymeric materials. For instance, a static charge build-up in the polymeric material can introduce a hazardous electrical discharge in the manufacturing environment. Also, static charge build-up can add to the cost of production, packaging, and shipping of polymeric materials and product. Another disadvantage of a build-up of static charge is that the surface of polymeric materials can experience static charge built-up, resulting in the tendency of the surface to collect dust.

Thermoplastic polymers, such as polyolefins, are particularly subject to accumulation of an electric charge because they are generally poor electrical conductors. In order to help prevent a charge build-up, the equipment used in the manufacturing of thermoplastic polymers is typically grounded to discharge any build-up of static charge in the polymer. However, the grounding of the equipment can be costly and complicated. Additionally, the grounding of the equipment can only temporarily help solve the static charge build-up problem in the thermoplastic polymer, since the polymer can continue to accumulate a static charge after leaving the grounded equipment.

For example, polyolefins, such as polypropylene, can be particularly subject to the build-up of a static charge in films, pellets, containers and other products. For instance, during the development of polyolefins, a charge can develop during the extrusion step of manufacture. This charge can result in a dangerous discharge of electricity. Further, the static charge may remain in the plastic products made from the polyolefin, which can attract dust onto the products, such as plastic bottles, giving an unsightly appearance of being on the shelf for a long period. For instance, films comprising polypropylene generally have a surface resistivity of about 10¹³ Ohms/square, indicating the build-up of a static charge in the film. In addition, films comprising polypropylene generally have an initial static decay average value between about 2.00 seconds and 9.00 seconds, indicating the time it takes for such films to dissipate an applied 5 kV static charge.

In the thermoplastic polymer industry, there have been many attempts to reduce the static charge build-up in polymeric materials. For instance, an anti-static material, or anti-stat, can be added to the surface of or incorporated into the polymeric material to reduce the build-up of a static charge. However, many current anti-stats for thermoplastic polymers known in the art have undesirable side effects, such as yellowing or hazing of the polymeric material.

There is currently a need for an anti-stat agent which can be incorporated into a thermoplastic polymer and can reduce or prevent the build-up of static charge without otherwise affecting the properties of the polymeric material or the products and films made with them.

### SUMMARY

In general, the present disclosure is directed to an anti-static agent that can be combined with a thermoplastic polymer for reducing static electricity.

In one embodiment, the present disclosure is directed to a polymer composition comprising:
a thermoplastic polymer; and
an anti-static agent present in the polymer composition in an amount sufficient to reduce surface resistivity, the anti-static agent having the following formula:

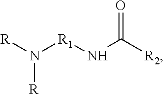

wherein R is CH₃(CH₂)ₓ, R₁ is (CH₂)_{y}, and R₂ is (CH₂)_{z}CH₃;

and wherein X and Z are independently from 0 to 20 and Y is from 1 to 20.

In one embodiment, the anti-static agent may comprise a quaternary compound. For instance, the anti-static agent may have the following formula:

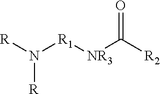

wherein R₃ is a group that produces a quaternary compound. R₃ may comprise an alkyl group or an aromatic group. When present as a quaternary compound, anions may be present, such as halide ions, such as chlorine ions.

In one embodiment, the anti-static agent may further comprise a polyglycerol ester. The polyglycerol ester may have one of the following formulas:

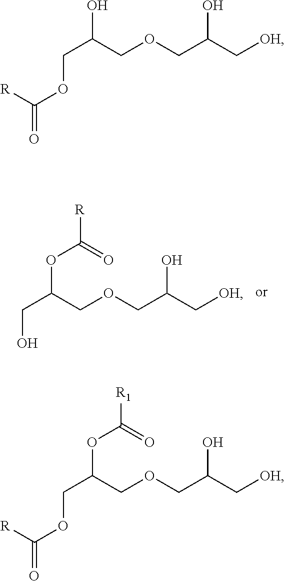

wherein R and R₁ are (CH₂)_{z}CH₃ and where z is from 0 to 16.

In another embodiment, the present disclosure is directed to an anti-static agent for a polymer composition. The anti-static agent may comprise a polyglycerol ester combined with the following:

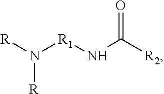

wherein R is CH₃(CH₂)ₓ, R₁ is (CH₂)_{y}, and R₂ is (CH₂)_{z}CH₃; and

wherein x is from 0 to 20, such as from 0 to 7, R₁ y is from 1 to 20, such as from 1 to 8, and z is from 0 to 20, such as from 0 to 16.

Other features and aspects of the present disclosure are discussed in greater detail below.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

Generally, in accordance with the present disclosure, an anti-stat agent is combined with a polymeric material to reduce the build-up of static charge in the polymeric material. The anti-stat agent is an amidoamine produced from the reaction of a fatty acid and an aminoamine. The fatty acid can be saturated or unsaturated, with a carbon chain of 8 to 18 carbons. The aminoamines can be Di C₁-C₈ amino C₁₋₈ amines. Additionally, the anti-stat agent can be a blend of the previously mentioned amidoamine with a polyglycerol ester (PGE) to create amidoamine/PGE blends with various blend ratios.

One embodiment of the present disclosure is directed toward a polymeric material and polymeric films and molded parts which are resistant to the build-up of electric charge, which can develop during or after the processing of the polymeric material whether by extrusion or by various types of molding processes or the packaging of the end polymeric products. In another embodiment, the present disclosure is generally directed toward processes and methods for reducing the build-up of electric charge in polymeric materials and the products made from the polymeric materials.

The amount of static charge build-up in a material can be measured by the surface resistivity of the material. Surface resistivity is a measure of the intensity of electrical current flowing over the surface of the material, quantifying the ability of the surface to conduct electricity in Ohms/square. Generally, an effective anti-static additive is one that will reduce the surface resistivity of the material to which it is applied or incorporated.

According to the present disclosure, the build-up of static charge in the polymeric material can be reduced by combining an anti-stat agent with the polymeric material. The anti-stat agent can be combined with the host polymeric material by any means known to one skilled in the art at any time during the production of the polymeric material or during production of films, containers, or other products formed from the polymeric material.

An anti-stat agent can be integrated internally into the polymeric material matrix during the production of the polymeric material. Generally, anti-stat agents incorporated into the polymeric material before or during the extrusion process are called internal anti-stat agents. For instance, the anti-stat agent can be simply mixed into the polymeric material after the polymerization of the polymeric material is complete. In one embodiment the anti-stat agent can be incorporated into the polymeric material during the extrusion process.

The polymeric material incorporating an internal anti-stat agent can be processed into any end product that would normally be produced by the polymeric material. For example, thermoplastic polymers incorporating an internal anti-stat agent can be made into films, pellets, fibers, containers, and any other end products normally manufactured from the thermoplastic polymer without substantially changing the properties and characteristics of the polymeric material or the products made with the polymeric material.

In another embodiment, the anti-stat agent of the present disclosure may be applied to the surface of the polymeric material after the extrusion process by any method known in the art, such as spraying, printing, dipping or wiping. Generally, an anti-stat agent applied after the extrusion process is referred to as an external anti-stat agent. However, external anti-stat agents can be particularly susceptible to removal from the polymeric surface. Thus, external anti-stat agents are generally not well adapted for long term reduction of static charge because of the ease in which they can be removed.

In many applications, the anti-stat agent of the present disclosure is substantially non-hazing to the host polymeric material. Without wishing to be bound by theory, an additive is non-hazing if the solubility of the additive is such that it does not interfere with the chemical structure or the refractive index of the extruded films or molded parts of the polymeric material. The films and molded parts of the polymeric material can remain transparent with the addition of a non-hazing additive.

Additionally, many additives to the thermoplastic polymer create an undesirable yellowing of the thermoplastic polymer, which interferes with the clarity of the thermoplastic polymer and the resulting thermoplastic polymer products. However, combining an anti-stat agent to the thermoplastic polymer according to the present disclosure does not substantially yellow or otherwise substantially interfere with the clarity of the thermoplastic polymer. Without wishing to be bound by theory, it is believed that to be a non-yellowing additive, the additive must be thermally stable at the extrusion and forming temperatures of the polymeric material so the additive does not chemically degrade, preventing any color formation of the additive.

Also, the anti-stat agent of the present disclosure can be migratory. A migratory additive is one that will migrate to the surface of the polymeric film or molded parts. If the migratory additive is removed from the surface by physical or chemical action, then the migratory additive will regenerate a new additive film on the surface of the film or molded parts. Generally, the additive will have two functional groups, one that is more soluble with the host polymeric material than the other. The less soluble component of the additive will tend to migrate toward the surface of the polymeric material, while the more soluble component of the anti-stat agent will tend to stay within the polymeric material matrix.

The rate an additive migrates can depend on a number of factors known to one skilled in the art, including the relative solubility of the additive and the polymeric material, the crystallinity of the polymeric material, and the concentration of the additive. Additionally, one skilled in the art can substantially control the migration rate of the additive by adjusting the additive's characteristics to change one of the rate factors.

According to the present disclosure, the polymeric material can be any plastic material that has the tendency to build-up a static charge, including but not limited to thermoplastic polymers. For instance, the polymeric material can comprise a polyolefin, such as polypropylene or polyethylene. In other embodiments, the polymeric material can comprise polyesters, polyamides, polystyrenes, polyethylenes, and/or polycarbonates. For example, the polymeric material can comprise polyethylene terephthalate, polybutylene terephthalate, or a polylactic acid or polylactide. In addition, the polymeric material can comprise low-density polyethylene, linear low-density polyethylene or high-density polyethylene.

As described above, the anti-stat agent of the present disclosure comprises a derivatized fatty acid. As used herein, a derivatized fatty acid is a fatty acid that has been reacted with at least one other compound. For example, the derivatized fatty acid can be reacted with a polyamine, such as a diamine, to form an amidoamine. Additionally, for instance, the amidoamine can be further combined with a polyglycerol ester (PGE), such as phosphorous pentoxide, polyphosphoric acid, or the like.

Fatty acids are carboxylic acids with a long aliphatic tail typically having the formula of

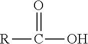

where R represents a hydrocarbon chain, either saturated or unsaturated. According to the present disclosure, The hydrocarbon chain of the fatty acid can be of any length, such as comprising from about 8 to about 18 carbons, for example from about 10 to about 16 carbons. Alternatively, in other embodiments, the hydrocarbon chain can comprise from about 12 carbons to about 14 carbons. For instance, in one particular embodiment, the fatty acid can have a hydrocarbon chain comprising 12 carbons.

The hydrocarbon chain of the fatty acid used to produce the anti-stat agent can be either saturated or unsaturated, including both monounsaturated and polyunsaturated fatty acids. A saturated carbon chain means that all the carbon to carbon bonds in the hydrocarbon chain are single bonds, allowing the maximum number of hydrogens to bond to each carbon, thus the chain is “saturated” with hydrogen atoms.

An unsaturated hydrocarbon chain means that the carbon chain contains at least one carbon to carbon double bond, thereby reducing the number of hydrogens present on the chain. A monounsaturated hydrocarbon chain contains one carbon to carbon double bond, while a polyunsaturated hydrocarbon chain contains at least two carbon to carbon double bonds.

Many fatty acids have common names, relating to their corresponding hydrocarbon chain, to describe the acid. The hydrocarbon chains can also be described by the number of carbon atoms present in the chain and the number and location of any double bonds present in the chain, represented by n:m^{Δp,p′,p″}, where n is the number of carbons in the hydrocarbon chain, m is the number of carbon to carbon double bonds in the chain, p is the location of the first double bond (if present), p′ is the location of the second double bond (if present), p″ is the location of the third double bond (if present), and so on.

Examples of saturated fatty acids that can be used to produce an anti-stat agent according to the present disclosure include, but are not limited to, caprylic acid (8:0), lauric acid (12:0), tridecylic acid (13:0), myristic acid (14:0), pentadecylic acid (15:0), cetylic acid (16:0, also known as palmitoleic acid), heptadecanoic acid (17:0), and stearic acid (18:0).

Examples of unsaturated fatty acids that can be used to produce an anti-stat agent according to the present disclosure include, but are not limited to, palmitoleic acid (16:1^{Δ9}), oleic acid (18:1^{Δ9}), linoleic acid (18,2^{Δ9,12}), conjugated linoleic acid (18:2^{Δ9,11}), linolenic acid (18:3^{Δ9,12,15}), and γ-linolenic acid (18:3^{Δ6,9,12}). The Δ^{X} nomenclature denotes each double bond, where the double bond is located on the xth carbon-carbon bond.

Also, in some embodiments, the hydrocarbon chain of the fatty molecule can comprise a reactive group. For instance, the hydrocarbon chain can comprise an acrylate group.

As described above, the anti-stat agent of the present disclosure can be the product of a reaction between a fatty acid and an aminoamine, which produces an amidoamine. For example, the aminoamine can be a polyamine such as a diamine. Examples of the diamine include, but are not limited to, dimethylaminopropylamine (DMAPA), hexamethylenediamine, diphenylethylenediamine, and diamimocyclohexane. The resulting product of the reaction between the fatty acid and polyamine can generally be represented, in one embodiment, by the following formula:

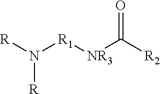

where R, R₁, and R₂ are alkyl groups. For example, R can have the composition CH₃(CH₂)ₓ, where x is from 0 to about 7, R₁ can have the composition (CH₂)_{y}, where y is from 1 to about 8, and R₂ can have the composition (CH₂)_{z}CH₃, where z is from 0 to about 16, such as from about 6 to about 14. In one particular embodiment, y can be about 3. In addition, R₂ may also contain unsaturation in the carbon chain. R₃ may comprise hydrogen or a group that produces a quaternary compound, such as a quaternary salt. R₃, for instance, may comprise an alkyl group, an aromatic group, and the like. In one embodiment, R₃ may include a benzyl group by reacting the amidoamine with a benzyl chloride. When R₃ produces a quaternary compound, anions may be present.

In one particular embodiment, the fatty acid may comprise about 40 to 80 percent by weight, such as from about 50 to 75 percent by weight, such as from about 60 to 70 percent by weight, the amidoamine. The diamine may comprise about 20 to 60 percent by weight, such as from about 25 to 50 percent by weight, such as from about 30 to 40 percent by weight, the amidoamine. For example, the amidoamine can be the product of the reaction between lauric acid and dimethylaminopropylamine.

The anti-stat agent previously described can be combined with the polymeric material such that the anti-stat agent comprises from about 0.01% to about 10% of by weight of the polymeric material, such as from about 0.1% to about 5% of the polymeric material. For instance, the anti-stat agent can comprise from about 0.1% to about 3.5% of the polymeric material, such as from about 0.1% to about 1% of the polymeric material. In one embodiment, the anti-stat agent can comprise about 0.5% by weight of the polymeric material. The anti-stat agent can be added to the polymeric material either alone or in combination with other additives or anti-stat agents.

In one embodiment, the previously described amidoamine can optionally be combined with a polyglycerol ester (PGE), to create amidoamine/PGE blends. The polyglycerol esters, for example, can have the following structures:

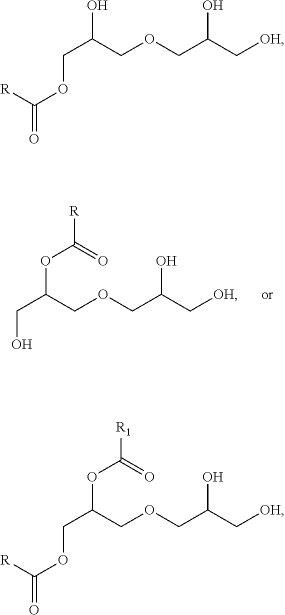

where R and R₁ are alkyl groups. For example, R and R₁ can have the composition (CH₂)_{z}CH₃, where z is from 0 to about 16. In addition, R and R₁ may contain polyunsaturation in the carbon chain.

The amidoamine can be combined with the PGE with an amidoamine to PGE blend ratio ranging from about 80:20 to about 20:80. For instance, the blend ratio can be from about 70:30 to about 30:70, such as from about 60:40 to about 40:60. In one particular embodiment, the blend ratio can be from about 55:45 to about 45:55, such as about 50:50.

In one embodiment, water can be associated with the anti-stat agent. Such as, in one embodiment, the polymeric material and anti-stat agent can be combined then exposed to a humid environment, allowing water vapor to associate with the polymeric material.

For instance, water can be associated with the anti-stat agent after the anti-stat agent has been incorporated into the polymeric material. For example, the anti-stat agent can be added to the polymeric material, then the polymeric material comprising the anti-stat agent can be molded into the final product, such as a container. Once the anti-stat agent is in the final product of the polymeric material, the anti-stat agent can migrate to the surface of the polymeric material where it can attract water molecules. For example, after extruding the anti-stat agent into the polymer, the anti-stat can migrate to the surface of the polymeric material where the anti-stat agent can attract water.

The polymeric material incorporating the anti-stat agent can be formed into any end product that would typically be produced from the particular polymeric material. For example, thermoplastic polymers incorporating the anti-stat agent of the present disclosure can be made into films or molded into any shape that is desired. For instance, thermoplastic polymers incorporating an anti-stat agent of the present disclosure can be made into films, pellets, fibers, containers, bottles, and other products.

In one embodiment, the anti-stat agent can be added to the polymeric material, such as mixed into the polymeric material in its resin form. Then, the polymeric material and the anti-stat agent mixture can be extruded, such as melt-blending, into a molded form. For instance, an amidoamine and/or an amidoamine/PGE blend can be added to thermoplastic polymers in its resin form. Then the mixture can be extruded into a film or a molded material, such as a container.

In another embodiment, the anti-stat agent of the present disclosure can be compounded with the polymeric material. For instance, a thermoplastic polymer resin and an anti-stat agent according to the present disclosure can be melt-blended together forming compounded pellets.

In another embodiment, the anti-stat agent can be added to the polymeric material during the formation of the films or molded parts and products, such as during the extrusion of the polymeric material. For instance, the anti-stat agent can be added to the thermoplastic polymer during the extrusion of the thermoplastic polymer into films or molded parts, such as containers, pellets, or the like.

In yet another embodiment, the anti-stat agent can be added to the polymeric material extrusion, molding, or the like. Application of the anti-stat agent to the polymeric material can be done through any means known in the art. Application methods include, but are not limited to, spraying, printing, dipping, or wiping.

In still another embodiment, the anti-stat agent can be added during formation of the thermoplastic polymer. For instance, the anti-stat agent may be added to the monomers that are used to produce the thermoplastic polymer.

In one embodiment, the anti-stat agent can be added to thermoplastic polymers in such a concentration that a final product made from the thermoplastic polymers anti-stat mixture, such as a film or molded part, comprises from about 0.1% to about 10% anti-stat agent by weight, such as about 0.1% to about 5%. For instance, in one embodiment, the thermoplastic polymers anti-stat mixture can comprise from about 0.1% to about 3.5% anti-stat agent by weight, such as from about 0.25% to about 2%, such as about 0.5%.

The final product produced from the polymeric material incorporating the anti-stat agent of the present disclosure can have a lower surface resistivity than that of the polymeric material without the anti-stat agent present. For example, polypropylene normally has a surface resistivity of about 10¹³ ohms/square. However, when polypropylene is mixed with an anti-stat agent according to the present disclosure, the resulting polypropylene and anti-stat agent material can have an initial surface resistivity of less than about 10¹² ohms/square, such as Less than about 10¹¹ ohms/square. After 90 days, the polymeric material can have a surface resistance of less than about 10¹⁰ ohms/square, such as less than about 10⁹ ohms/square.

When the anti-stat agent is combined with the thermoplastic polymer, the initial surface resistivity of the polymer can decrease by at least about 3%, such as at least about 5%, such as at least about 7%, such as at least about 10%, such as at least about 12%, such as even less than about 15%. The initial surface resistivity can decrease by up to about 90%, such as up to about 80%, such as up to about 70%, such as up to about 60%, such as up to about 50%. After 90 days, the same reductions in surface resistivity as described above can occur. For instance, after 90 days, the surface resistivity may be decreased by at least about 3%, such as at least about 5%, such as at least about 7%, such as at least about 10%, such as at least about 12%, such as at least about 15%, such as even less than about 20% in comparison to the same thermoplastic material not containing the anti-stat agent.

Additionally, the final product produced from the polymeric material incorporating the anti-stat agent of the present disclosure can have a lower static decay value than that of the polymeric material without the anti-stat agent present. For example, polypropylene generally has a static decay average value of between 0.1 and 3.00 seconds. However, when polypropylene is mixed with an anti-stat agent according to the present disclosure, the resulting polypropylene and anti-stat agent material can have an initial static decay average value less than about 2.00 seconds, such as less than about 1.00 seconds. For instance, in one embodiment, the polypropylene and anti-stat agent material can have an initial static decay value less than about 0.15 seconds. Furthermore, in yet another embodiment, the resulting polypropylene and anti-stat agent material can have an initial static average value of less than about 0.05 seconds, such as less than about 0.02 seconds.

The present disclosure may be better understood with reference to the following examples.

### EXAMPLES

The controls and compare polymers provided in the following examples are used to show the effect of the anti-stat agent on the polymers static properties. In the following tables, the control is a polymer that does not contain an antistatic agent. Unless otherwise stated, Compare 1 is a polymer with a natural amine polyoxyethylene additive and Compare 2 is a polymer with a synthetic amine polyoxyethylene additive. The polymers of Compare 1 and 2 correspond to the polymer of the example it is being compared to. For instance, in Example 1, the polymer of Compare 1 and 2 is a polypropylene copolymer obtained from Flints Hills Resources (Longview, Tex.). An asterisk (*) indicates that a sample had no antistatic properties and was therefore not testable.

In the following tables, the static resistivity was determined by ASTM Test D257, grounded plane, and EOS/ESD 11.11 insulated plane standard test. The static decay average value was determined by NFPA Test 99, at a 10% cut off, and the Mil-PRF-81705D low humidity test method. The total amine value was determined by AMV Test 001. The test specimens comprised a film having dimensions of 3.5 inches×6.5 inches. The film had a thickness of from about 0.1 mm to about 0.5 mm.

### Example No. 1 & 3

Example 1 comprises a polypropylene copolymer obtained from Flint Hills Resources (Longview, Tex.). The polymer is mixed with 0.1% by weight of the anti-stat additive agent, which is an amidoamine produced from the reaction of a C₁₂₋₁₄ saturated fatty acid reacted with Di C₁ amino C₃ amine.

Example 3 comprises a polypropylene copolymer obtained from Flint Hills Resources (Longview, Tex.). The polymer is mixed with 0.1% by weight of the anti-stat additive agent, which is an amidoamine produced from the reaction of a C₁₂₋₁₄ saturated fatty acid reacted with Di C₁ amino C₃ amine blended with a C₈₋₁₈ saturated polyglycerol ester at a 50:50 blend ratio.

Surface Resistivity (ohms/square)

| Day | Control | Compare 1 | Compare 2 | Example 1 | Example 3 |
|---|---|---|---|---|---|
| Initial | 10\hat{ }14 | 10\hat{ }14 | 10\hat{ }15 | 10\hat{ }13 | 10\hat{ }11 |
| Week 1 | 10\hat{ }14 | 10\hat{ }13 | 10\hat{ }12 | 10\hat{ }12 | 10\hat{ }12 |
| Week 2 | 10\hat{ }14 | 10\hat{ }13 | 10\hat{ }12 | 10\hat{ }12 | 10\hat{ }12 |
| 1 Month | 10\hat{ }14 | 10\hat{ }13 | 10\hat{ }12 | 10\hat{ }11 | 10\hat{ }11 |
| 2 Months | 10\hat{ }14 | 10\hat{ }13 | 10\hat{ }12 | 10\hat{ }11 | 10\hat{ }12 |
| 3 Months | 10\hat{ }14 | 10\hat{ }12 | 10\hat{ }12 | 10\hat{ }11 | 10\hat{ }11 |
| 4 Months | 10\hat{ }13 | 10\hat{ }13 | 10\hat{ }11 | 10\hat{ }10 | 10\hat{ }10 |

Static Decay Average Value (seconds)

| Day | Control | Compare 1 | Compare 2 | Example 1 | Example 3 |
|---|---|---|---|---|---|
| Initial | 2.45 | * | 9.86 | * | 0.12 |
| Week 1 | * | * | 1.54 | 0.54 | 0.19 |
| Week 2 | * | 7.34 | 0.91 | 0.24 | 0.38 |
| 1 Month | * | 2.16 | 0.83 | 0.05 | 0.10 |
| 2 Months | * | 2.86 | 1.43 | 0.04 | 0.12 |
| 3 Months | * | 0.46 | 0.57 | 0.03 | 0.04 |
| 4 Months | * | 0.27 | 0.14 | 0.02 | 0.02 |

### Example No. 2 & 4

Example 2 comprises a polypropylene copolymer obtained from Flint Hills Resources (Longview, Tex.). The polymer is mixed with 0.5% by weight of the anti-stat additive agent, which is an amidoamine produced from the reaction of a C₁₂₋₁₄ saturated fatty acid reacted with Di C₁ amino C₃ amine.

Example 4 comprises a polypropylene copolymer obtained from Flint Hills Resources (Longview, Tex.). The polymer is mixed with 0.1% by weight of the anti-stat additive agent, which is an amidoamine produced from the reaction of a C₁₂₋₁₄ saturated fatty acid reacted with Di C₁ amino C₃ amine blended with a C₈₋₁₈ saturated polyglycerol ester at a 50:50 blend ratio.

Surface Resistivity (ohms/square)

| Day | Control | Compare 1 | Compare 2 | Example 2 | Example 4 |
|---|---|---|---|---|---|
| Initial | 10\hat{ }14 | 10\hat{ }11 | 10\hat{ }14 | 10\hat{ }14 | 10\hat{ }11 |
| Week 1 | 10\hat{ }14 | 10\hat{ }11 | 10\hat{ }12 | 10\hat{ }10 | 10\hat{ }11 |
| Week 2 | 10\hat{ }14 | 10\hat{ }11 | 10\hat{ }12 | 10\hat{ }10 | 10\hat{ }11 |
| 1 Month | 10\hat{ }14 | 10\hat{ }11 | 10\hat{ }11 | 10\hat{ }10 | 10\hat{ }10 |
| 2 Months | 10\hat{ }14 | 10\hat{ }11 | 10\hat{ }11 | 10\hat{ }10 | 10\hat{ }10 |
| 3 Months | 10\hat{ }14 | 10\hat{ }10 | 10\hat{ }11 | 10\hat{ }9 | 10\hat{ }10 |
| 4 Months | 10\hat{ }13 | 10\hat{ }10 | 10\hat{ }10 | 10\hat{ }9 | 10\hat{ }9 |

Static Decay Average Value (seconds)

| Day | Control | Compare 1 | Compare 2 | Example 2 | Example 4 |
|---|---|---|---|---|---|
| Initial | 2.45 | 0.59 | * | 0.13 | 0.02 |
| Week 1 | * | 0.15 | 1.88 | 0.03 | 0.05 |
| Week 2 | * | 0.06 | 0.29 | 0.02 | 0.03 |
| 1 Month | * | 0.09 | 0.09 | 0.01 | 0.03 |
| 2 Months | * | 0.08 | 0.09 | 0.01 | 0.02 |
| 3 Months | * | 0.03 | 0.05 | 0.01 | 0.02 |
| 4 Months | * | 0.02 | 0.04 | 0.06 | 0.01 |

### Example No. 5

Example 5 comprises a polypropylene copolymer obtained from Flint Hills Resources (Longview, Tex.). The polymer is mixed with 0.1% by weight of the anti-stat additive agent, which is an amidoamine produced from the reaction of a C₁₂₋₁₄ saturated fatty acid reacted with Di C₁ amino C₃ amine blended with a C₈₋₁₈ saturated polyglycerol ester at a 30:70 blend ratio,

Surface Resistivity (ohms/square)

| Day | Control | Compare 1 | Compare 2 | Example 5 |
|---|---|---|---|---|
| Initial | 10\hat{ }14 | 10\hat{ }14 | 10\hat{ }15 | 10\hat{ }14 |
| Week 1 | 10\hat{ }14 | 10\hat{ }13 | 10\hat{ }12 | 10\hat{ }14 |
| Week 2 | 10\hat{ }14 | 10\hat{ }13 | 10\hat{ }12 | 10\hat{ }13 |
| 1 Month | 10\hat{ }14 | 10\hat{ }13 | 10\hat{ }12 | 10\hat{ }13 |
| 2 Months | 10\hat{ }14 | 10\hat{ }13 | 10\hat{ }12 | 10\hat{ }12 |
| 3 Months | 10\hat{ }14 | 10\hat{ }12 | 10\hat{ }12 | 10\hat{ }11 |
| 4 Months | 10\hat{ }13 | 10\hat{ }13 | 10\hat{ }11 | — |

Static Decay Average Value (seconds)

| Day | Control | Compare 1 | Compare 2 | Example 5 |
|---|---|---|---|---|
| Initial | 2.45 | * | 9.86 | * |
| Week 1 | * | * | 1.54 | * |
| Week 2 | * | 7.34 | 0.91 | 3.56 |
| 1 Month | * | 2.16 | 0.83 | 0.49 |
| 2 Months | * | 2.86 | 1.43 | 0.17 |
| 3 Months | * | 0.46 | 0.57 | 0.15 |
| 4 Months | * | 0.27 | 0.14 | — |

### Example No. 6

Example 5 comprises a polypropylene copolymer obtained from Flint Hills Resources (Longview, Tex.). The polymer is mixed with 0.5% by weight of the anti-stat additive agent, which is an amidoamine produced from the reaction of a C₁₂₋₁₄ saturated fatty acid reacted with Di C₁ amino C₃ amine blended with a C₈₋₁₈ saturated polyglycerol ester at a 45:55 blend ratio.

Surface Resistivity (ohms/square)

| Day | Control | Compare 1 | Compare 2 | Example 6 |
|---|---|---|---|---|
| Initial | 10\hat{ }14 | 10\hat{ }11 | 10\hat{ }14 | 10\hat{ }12 |
| Week 1 | 10\hat{ }14 | 10\hat{ }11 | 10\hat{ }12 | 10\hat{ }11 |
| Week 2 | 10\hat{ }14 | 10\hat{ }11 | 10\hat{ }12 | 10\hat{ }11 |
| 1 Month | 10\hat{ }14 | 10\hat{ }11 | 10\hat{ }11 | 10\hat{ }10 |
| 2 Months | 10\hat{ }14 | 10\hat{ }11 | 10\hat{ }11 | 10\hat{ }10 |
| 3 Months | 10\hat{ }14 | 10\hat{ }10 | 10\hat{ }11 | 10\hat{ }10 |
| 4 Months | 10\hat{ }13 | 10\hat{ }10 | 10\hat{ }10 | — |

Static Decay Average Value (seconds)

| Day | Control | Compare 1 | Compare 2 | Example 6 |
|---|---|---|---|---|
| Initial | 2.45 | 0.59 | * | 0.08 |
| Week 1 | * | 0.15 | 1.88 | 0.07 |
| Week 2 | * | 0.06 | 0.29 | 0.04 |
| 1 Month | * | 0.09 | 0.09 | 0.02 |
| 2 Months | * | 0.08 | 0.09 | 0.02 |
| 3 Months | * | 0.03 | 0.05 | 0.01 |
| 4 Months | * | 0.02 | 0.04 | — |

### Example No. 7 & 8

Example 7 comprises a polypropylene homopolymer obtained from Lyondell Basell (Houston, Tex.). The polymer is mixed with 0.1% by weight of the anti-stat additive agent, which is an amidoamine produced from the reaction of a C₁₂₋₁₄ saturated fatty acid reacted with Di C₁ amino C₃ amine.

Example 8 comprises a polypropylene homopolymer obtained from Lyondell Basell (Houston, Tex.). The polymer is mixed with 0.1% by weight of the anti-stat additive agent, which is an amidoamine produced from the reaction of a C₁₂₋₁₄ saturated fatty acid reacted with Di C₁ amino C₃ amine blended with a C₈₋₁₈ saturated polyglycerol ester at a 50:50 blend ratio.

Surface Resistivity (ohms/square)

| Day | Control | Compare 1 | Compare 2 | Example 7 | Example 8 |
|---|---|---|---|---|---|
| Initial | 10\hat{ }14 | 10\hat{ }12 | 10\hat{ }13 | 10\hat{ }13 | 10\hat{ }11 |
| Week 1 | 10\hat{ }13 | 10\hat{ }13 | 10\hat{ }13 | 10\hat{ }12 | 10\hat{ }13 |
| Week 2 | 10\hat{ }13 | 10\hat{ }13 | 10\hat{ }12 | 10\hat{ }13 | 10\hat{ }10 |
| 1 Month | 10\hat{ }14 | 10\hat{ }13 | 10\hat{ }13 | 10\hat{ }13 | 10\hat{ }13 |
| 2 Months | 10\hat{ }15 | 10\hat{ }14 | 10\hat{ }13 | 10\hat{ }13 | 10\hat{ }10 |
| 3 Months | 10\hat{ }16 | 10\hat{ }11 | 10\hat{ }12 | 10\hat{ }11 | 10\hat{ }12 |
| 4 Months | 10\hat{ }15 | 10\hat{ }10 | 10\hat{ }14 | 10\hat{ }11 | 10\hat{ }12 |
| 5 Months | 10\hat{ }15 | 10\hat{ }13 | 10\hat{ }14 | 10\hat{ }12 | 10\hat{ }13 |

Static Decay Average Value (seconds)

| Day | Control | Compare 1 | Compare 2 | Example 7 | Example 8 |
|---|---|---|---|---|---|
| Initial | * | 0.17 | 0.12 | 0.13 | 0.03 |
| Week 1 | * | 0.90 | 0.85 | 0.82 | 0.40 |
| Week 2 | * | 2.86 | 0.07 | 1.35 | 0.29 |
| 1 Month | * | 0.82 | 1.30 | 1.39 | 0.87 |
| 2 Months | * | 1.64 | 1.81 | 1.21 | 0.79 |
| 3 Months | * | 0.34 | 0.50 | 0.16 | 0.54 |
| 4 Months | * | 2.56 | * | 0.58 | 2.79 |
| 5 Months | * | 2.99 | 4.47 | 0.53 | * |

### Example No. 9 & 10

Example 9 comprises a polypropylene homopolymer obtained from Lyondell Basell (Houston, Tex.). The polymer is mixed with 0.5% by weight of the anti-stat additive agent, which is an amidoamine produced from the reaction of a C₁₂₋₁₄ saturated fatty acid reacted with Di C₁ amino C₃ amine.

Example 10 comprises a polypropylene homopolymer obtained from Lyondell Basell (Houston, Tex.). The polymer is mixed with 0.5% by weight of the anti-stat additive agent, which is an amidoamine produced from the reaction of a C₁₂₋₁₄ saturated fatty acid reacted with Di C₁ amino C₃ amine blended with a C₈₋₁₈ saturated polyglycerol ester at a 50:50 blend ratio.

Surface Resistivity (ohms/square)

| | Con- | | | | |
|---|---|---|---|---|---|
| Day | trol | Compare 1 | Compare 2 | Example 9 | Example 10 |
| Initial | 10\hat{ }14 | 10\hat{ }11 | 10\hat{ }13 | 10\hat{ }11 | 10\hat{ }10 |
| Week 1 | 10\hat{ }13 | 10\hat{ }11 | 10\hat{ }12 | 10\hat{ }11 | 10\hat{ }10 |
| Week 2 | 10\hat{ }13 | 10\hat{ }11 | 10\hat{ }12 | 10\hat{ }11 | 10\hat{ }10 |
| 1 Month | 10\hat{ }14 | 10\hat{ }11 | 10\hat{ }12 | 10\hat{ }10 | 10\hat{ }10 |
| 2 Months | 10\hat{ }15 | 10\hat{ }11 | 10\hat{ }12 | 10\hat{ }11 | 10\hat{ }11 |
| 3 Months | 10\hat{ }16 | 10\hat{ }10 | 10\hat{ }11 | 10\hat{ }10 | 10\hat{ }10 |
| 4 Months | 10\hat{ }15 | 10\hat{ }11 | 10\hat{ }13 | 10\hat{ }11 | 10\hat{ }11 |
| 5 Months | 10\hat{ }15 | 10\hat{ }11 | 10\hat{ }12 | 10\hat{ }10 | 10\hat{ }11 |

Static Decay Average Value (seconds)

| | | | | | Example |
|---|---|---|---|---|---|
| Day | Control | Compare 1 | Compare 2 | Example 9 | 10 |
| Initial | * | 0.02 | 0.06 | 0.02 | 0.01 |
| Week 1 | * | 0.05 | 0.12 | 0.05 | 0.03 |
| Week 2 | * | 0.07 | 0.15 | 0.04 | 0.03 |
| 1 Month | * | 0.05 | 0.16 | 0.07 | 0.03 |
| 2 Months | * | 0.15 | 0.24 | 0.12 | 0.11 |
| 3 Months | * | 0.03 | 0.05 | 0.02 | 0.03 |
| 4 Months | * | 0.11 | 0.22 | 0.05 | 0.13 |
| 5 Months | * | 0.11 | 0.21 | 0.04 | 0.14 |

### Example No. 11 & 12

Example 11 comprises a polyactide, PLA 4042D, obtained from NatureWorks (Minnetonka, Minn.). The polymer is mixed with 0.5% by weight of the anti-stat additive agent, which is an amidoamine produced from the reaction of a C₁₂₋₁₄ saturated fatty acid reacted with Di C₁ amino C₃ amine blended with a C₈₋₁₈ saturated polyglycerol ester at a 50:50 blend ratio.

Example 12 comprises a polyactide, PLA 4042D, obtained from NatureWorks (Minnetonka, Minn.). The polymer is mixed with 0.5% by weight of the anti-stat additive agent, which is an amidoamine produced from the reaction of a C₁₆₋₁₈ saturated fatty acid reacted with Di C₁ amino C₃ amine blended with a C₁₆₋₁₈ saturated polyglycerol ester at a 50:50 blend ratio.

Surface Resistivity (ohms/square)

| | | Compare | Compare | | |
|---|---|---|---|---|---|
| Day | Control | 1 | 2 | Example 11 | Example 12 |
| Initial | 10\hat{ }15 | — | 10\hat{ }15 | 10\hat{ }13 | 10\hat{ }13 |
| Week 1 | 10\hat{ }15 | — | 10\hat{ }13 | 10\hat{ }15 | 10\hat{ }13 |
| Week 2 | 10\hat{ }13 | — | 10\hat{ }13 | 10\hat{ }12 | 10\hat{ }13 |
| 1 Month | 10\hat{ }16 | — | 10\hat{ }15 | 10\hat{ }12 | 10\hat{ }13 |
| 2 Months | 10\hat{ }15 | — | 10\hat{ }15 | 10\hat{ }12 | 10\hat{ }12 |
| 3 Months | — | — | — | — | — |
| 4 Months | — | — | — | — | — |

Static Decay Average Value (seconds)

| | | Compare | Compare | | |
|---|---|---|---|---|---|
| Day | Control | 1 | 2 | Example 11 | Example 12 |
| Initial | * | — | * | 0.61 | * |
| Week 1 | * | — | * | 0.16 | 2.86 |
| Week 2 | * | — | * | 0.18 | 2.30 |
| 1 Month | * | — | * | 0.39 | 3.23 |
| 2 Months | * | — | * | 0.91 | 6.05 |
| 3 Months | — | — | — | — | — |
| 4 Months | — | — | — | — | — |

### Example No. 13 & 14

Example 13 comprises a polypropylene random copolymer obtained from LyondellBasell (Houston, Tex.), under the trade name Pro-fax SR549. The polymer is mixed with 0.1% by weight of the anti-stat additive agent, which is an amidoamine produced from the reaction of a C₁₈ unsaturated fatty acid reacted with Di C₁ amino C₃ amine blended with a C₁₈ polyunsaturated polyglycerol ester at a 50:50 blend ratio.

Example 14 comprises a polypropylene random copolymer obtained from LyondellBasell (Houston, Tex.), under the trade name Pro-fax SR549. The polymer is mixed with 0.1% by weight of the anti-stat additive agent, which is an amidoamine produced from the reaction of a C₁₈ unsaturated fatty acid reacted with Di C₁ amino C₃ amine.

Surface Resistivity (ohms/square)

| | | Compare | Compare | | |
|---|---|---|---|---|---|
| Day | Control | 1 | 2 | Example 13 | Example 14 |
| Initial | 10\hat{ }13 | 10\hat{ }11 | 10\hat{ }11 | 10\hat{ }11 | 10\hat{ }10 |
| Week 1 | 10\hat{ }12 | 10\hat{ }11 | 10\hat{ }10 | 10\hat{ }11 | 10\hat{ }10 |
| Week 2 | 10\hat{ }12 | 10\hat{ }11 | 10\hat{ }10 | 10\hat{ }11 | 10\hat{ }10 |
| 1 Month | — | — | — | — | — |
| 2 Months | 10\hat{ }13 | 10\hat{ }11 | 10\hat{ }11 | 10\hat{ }11 | 10\hat{ }10 |
| 3 Months | — | — | — | — | — |
| 4 Months | — | — | — | — | — |

Static Decay Average Value (seconds)

| | | Compare | Compare | | |
|---|---|---|---|---|---|
| Day | Control | 1 | 2 | Example 13 | Example 14 |
| Initial | 1.61 | 0.08 | 0.15 | 0.05 | 0.03 |
| Week 1 | 0.35 | 0.04 | 0.08 | 0.03 | 0.02 |
| Week 2 | 0.41 | 0.05 | 0.10 | 0.04 | 0.04 |
| 1 Month | — | — | — | — | — |
| 2 Months | 3.97 | 0.06 | 0.10 | 0.06 | 0.04 |
| 3 Months | — | — | — | — | — |
| 4 Months | — | — | — | — | — |

### Example No. 15 & 16

Example 15 comprises a polypropylene random copolymer obtained from LyondellBasell (Houston, Tex.), under the trade name Pro-fax SR549. The polymer is mixed with 0.5% by weight of the anti-stat additive agent, which is an amidoamine produced from the reaction of a C₁₆₋₁₈ saturated fatty acid reacted with Di C₁ amino C₃ amine blended with a C₁₋₈ saturated polyglycerol ester at a 50:50 blend ratio.

Example 16 comprises a polypropylene random copolymer obtained from LyondellBasell (Houston, Tex.), under the trade name Pro-fax SR549. The polymer is mixed with 0.1% by weight of the anti-stat additive agent, which is an amidoamine produced from the reaction of a C₁₆₋₁₈ saturated fatty acid reacted with Di C₁ amino C₃ amine.

Surface Resistivity (ohms/square)

| | | Compare | Compare | | |
|---|---|---|---|---|---|
| Day | Control | 1 | 2 | Example 15 | Example 16 |
| Initial | 10\hat{ }13 | 10\hat{ }11 | 10\hat{ }11 | 10\hat{ }10 | 10\hat{ }11 |
| Week 1 | 10\hat{ }12 | 10\hat{ }11 | 10\hat{ }10 | 10\hat{ }10 | 10\hat{ }10 |
| Week 2 | 10\hat{ }12 | 10\hat{ }11 | 10\hat{ }10 | 10\hat{ }10 | 10\hat{ }10 |
| 1 Month | — | — | — | — | — |
| 2 Months | 10\hat{ }13 | 10\hat{ }11 | 10\hat{ }11 | 10\hat{ }10 | 10\hat{ }10 |
| 3 Months | — | — | — | — | — |
| 4 Months | — | — | — | — | — |

Static Decay Average Value (seconds)

| | | Compare | Compare | | |
|---|---|---|---|---|---|
| Day | Control | 1 | 2 | Example 15 | Example 16 |
| Initial | 1.61 | 0.08 | 0.15 | 0.01 | 0.01 |
| Week 1 | 0.35 | 0.04 | 0.08 | 0.01 | 0.01 |
| Week 2 | 0.41 | 0.05 | 0.10 | 0.03 | 0.03 |
| 1 Month | — | — | — | — | — |
| 2 Months | 3.97 | 0.06 | 0.10 | 0.01 | 0.01 |
| 3 Months | — | — | — | — | — |
| 4 Months | — | — | — | — | — |

### Example No. 17 & 18

Example 17 comprises a compounded polypropylene copolymer obtained from Flint Hills Resources (Longview, Tex.). The polymer is created through a compounded material-degradation study starting with extrusion of a 1% add of the antistatic additive agent consisting of amidoamine produced from the reaction of a C₁₋₂ saturated fatty acid reacted with Di C₁ amino C₃ amine to the copolymer. The copolymer is then extruded 2 more ties, first reducing the level of the amidoamine to 0.5% by weight with additional copolymer, and a second time reducing the level of the amidoamine to a final concentration of 0.1% with additional copolymer.

Example 18 comprises a polypropylene copolymer obtained from Flint Hills Resources (Longview, Tex.). The polymer is created through a compounded material-degradation study starting with extrusion of a 1% add of the antistatic additive agent consisting of amidoamine produced from the reaction of a C₁₋₂ saturated fatty acid reacted with Di C₁ amino Cₐ amine blended with a C₁₋₂ saturated polyglycerol ester at a 50:50 blend ratio to the copolymer. The copolymer is then extruded 2 more ties, first reducing the level of the amidoamine to 0.5% by weight with additional copolymer, and a second time reducing the level of the amidoamine to a final concentration of 0.1% with additional copolymer.

Compare 1 in the study of Examples 17 & 18 is a quaternary ammonium chloride.

Surface Resistivity (ohms/square)

| | | Compare | Compare | | |
|---|---|---|---|---|---|
| Day | Control | 1 | 2 | Example 17 | Example 18 |
| Initial | 10\hat{ }14 | 10\hat{ }10 | 10\hat{ }12 | 10\hat{ }10 | 10\hat{ }13 |
| Week 1 | 10\hat{ }14 | 10\hat{ }10 | 10\hat{ }11 | 10\hat{ }10 | 10\hat{ }12 |
| Week 2 | 10\hat{ }14 | 10\hat{ }11 | 10\hat{ }11 | 10\hat{ }12 | 10\hat{ }12 |
| 1 Month | 10\hat{ }14 | 10\hat{ }12 | 10\hat{ }11 | 10\hat{ }11 | 10\hat{ }12 |
| 2 Months | — | — | — | — | — |
| 3 Months | — | — | — | — | — |
| 4 Months | 10\hat{ }13 | 10\hat{ }13 | 10\hat{ }13 | 10\hat{ }11 | 10\hat{ }11 |

Static Decay Average Value (seconds)

| | | Compare | Compare | | |
|---|---|---|---|---|---|
| Day | Control | 1 | 2 | Example 17 | Example 18 |
| Initial | * | 0.03 | 2.04 | 0.02 | 2.05 |
| Week 1 | * | 0.04 | 0.97 | 0.03 | 0.71 |
| Week 2 | * | 0.06 | 1.34 | 0.03 | 0.91 |
| 1 Month | * | 0.06 | 0.80 | 0.02 | 0.24 |
| 2 Months | — | — | — | — | — |
| 3 Months | — | — | — | — | — |
| 4 Months | * | 1.78 | 1.38 | 0.09 | 0.29 |

### Example No. 19 & 20

Example 19 comprises a polysterene obtained from Amco (product number 74A12000A-30). The polymer is mixed with 3.5% by weight of the anti-stat additive agent, which is an amidoamine produced from the reaction of a C₁₋₂ saturated fatty acid reacted with Di C₁ amino C₃ amine blended with a C₁₂ saturated polyglycerol ester at a 30:70 blend ratio.

Example 20 comprises a polysterene obtained from Amco (product number 74A12000A-30). The polymer is mixed with 3.5% by weight of the anti-stat additive agent, which is a C₁₂ saturated polyglycerol ester.

Compare 1 in the study of Examples 19 & 20 is a diglycerin laurate.

Static Decay Average Value (seconds)

| Day | Control | Compare 1 | Example 19 | Example 20 |
|---|---|---|---|---|
| Initial | * | 0.73 | 0.06 | 0.18 |
| Week 1 | * | 0.31 | 0.41 | 0.28 |
| Week 2 | * | 0.02 | 0.08 | 0.07 |
| 1 Month | * | 0.07 | 0.07 | 0.07 |
| 2 Months | * | 0.12 | 0.30 | 0.23 |
| 3 Months | * | — | 0.06 | 0.07 |
| 4 Months | * | — | 0.10 | 0.14 |

### Example No. 21

Example 21 comprises a high-density polyethylene obtained from NOVA Chemicals Sclair (Moon Township, Pa.), under the trade name NOVA 2909 HDPE. The polymer is mixed with 0.2% by weight of the anti-stat additive agent, which is an amidoamine produced from the reaction of a C₁₋₂ saturated fatty acid reacted with Di C₁ amino C₃ amine blended with a C₁₋₂ saturated polyglycerol ester at a 50:50 blend ratio.

Compare 1 in the study of Example 21 is a synthetic amine polyester olefin.

Surface Resistivity (ohms/square)

| Day | Control | Compare 1 | Example 20 |
|---|---|---|---|
| Initial | 10\hat{ }12 | 10\hat{ }11 | 10\hat{ }11 |
| Week 1 | 10\hat{ }13 | 10\hat{ }11 | 10\hat{ }10 |

Static Decay Average Value (seconds)

| Day | Control | Compare 1 | Example 20 |
|---|---|---|---|
| Initial | * | 0.16 | 0.02 |
| Week 1 | * | 0.14 | 0.02 |

### Example No. 22

Example 22 comprises a polypropylene copolymer obtained from Flint Hills Resources (Longview, Tex.). The polymer is mixed with 0.1% by weight of the anti-stat additive agent, which is an amidoamine produced from the reaction of a C₁₋₂ saturated fatty acid reacted with Di C₁ amino C₃ amine blended with a C₁₋₂ saturated polyglycerol ester at various ratios.

The total amine value is the quantity (mL) of hydrochloric acid (HCl) necessary to neutralize the total amount of primary, secondary, and tertiary amines in a sample. It is defined as the number of milligrams of potassium hydroxide equivalent to 1 gram of sample, and expressed as milligrams of potassium hydroxide (KOH) per gram of sample, mgKOH/g. A representative sample is dissolved in a suitable solvent, usually isopropyl alcohol, and titrated with hydrochloric acid (0.1N or 0.5N). The colorimetric endpoint is determined by addition of a bromophenol blue indicator solution and observing a color change from blue to yellow. The potentiometric endpoint is determined by a pH endpoint of 3.2-3.4. The total amine value is calculated from the mL's of HCL titrated and reported as mgKOH/g. The following table shows various ratios of amidoamine/polyglycerol ester to compare amine values and corresponding static decay results.

Blend ratio is reported as a ratio of amidoamine/polyglycerol ester.

| | | Static Decay Average Value (seconds) | | | |
|---|---|---|---|---|---|
| Sample | Amine value | Initial | 1 Month | 2 Months | 3 Months |
| Compare 1 | 192 | * | 2.16 | 0.46 | 0.27 |
| Compare 2 | 187 | 9.86 | 0.83 | 0.57 | 0.14 |
| 30/70 blend | 59 | * | 3.56 | 0.49 | 0.17 |
| 35/65 blend | 70 | * | 0.90 | 0.47 | 0.14 |
| 40/60 blend | 80 | * | 1.94 | 0.64 | 0.40 |
| 45/55 blend | 93 | * | 0.24 | 0.07 | 0.04 |
| 50/50 blend | 106 | 0.12 | 0.10 | 0.04 | 0.02 |

### Example 23

Example 23: Polypropylene copolymer with 0.1% of the antistatic additive agent comprising amidoamine produced from the reaction of a C₁₋₂ saturated fatty acid reacted with Di C₁ amino C₃ amine reacted further with Benzyl Chloride to produce a quaternary compound. See diagram of reaction product below, and see Surface Resistivity and Static Decay results in Tables 27 and 28. The control below is the polymer only.

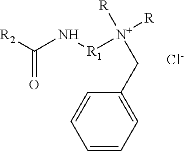

TABLE 27
Surface Resistivity Average Value

| | Control | Example 23 |
|---|---|---|
| Day | Side 1 | Side 1 |
| 1 | 10\hat{ }14 | 10\hat{ }11 |
| 8 | 10\hat{ }14 | 10\hat{ }10 |
| 18 | 10\hat{ }14 | 10\hat{ }11 |
| 25 | 10\hat{ }14 | 10\hat{ }11 |
| 32 | 10\hat{ }14 | 10\hat{ }11 |

TABLE 28
Static Decay Average Value

| Day | Control | Example 23 |
|---|---|---|
| 1 | * | 0.04 |
| 8 | * | 0.03 |
| 18 | * | 0.05 |
| 25 | * | 0.05 |
| 32 | * | 0.05 |

* indicates that the sample was not testable (no antistatic properties)

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

What is claimed:

1. A polymer composition comprising:
a thermoplastic polymer; and
an anti-static agent present in the polymer composition in an amount sufficient to reduce surface resistivity, the anti-static agent having the following formula: 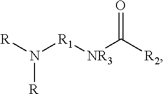
wherein R is CH₃(CH₂)ₓ, R₁ is (CH₂)_{y}, and R₂ is (CH₂)_{z}CH₃;
and wherein X and Z are independently from 0 to 20 and Y is from 1 to 20;
and wherein R₃ is H or a group that produces a quaternary compound.

2. The polymeric material of claim 1 with anti-static agent formula, wherein x is from 0 to 7, R₁ y is from 1 to 8, and z is from 0 to 16.

3. The polymeric material of claim 1, wherein the anti-static agent comprises a dimethylaminopropylamine.

4. The polymeric material of claim 1, wherein the anti-static agent further comprises a polyglycerol ester.

5. The polymeric material of claim 4, wherein the polyglycerol ester has one of the following formulas: 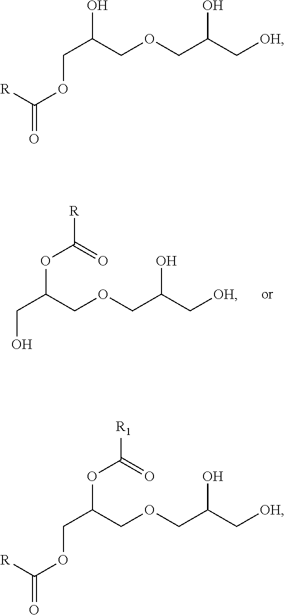
wherein R and R₁ are (CH₂)_{z}CH₃ and where z is from 0 to 16.

6. The polymeric material of claim 4, wherein the anti-static agent contains an amidoamine to polyglycerol ester ratio ranging from 80:20 to 20:80.

7. The polymeric material of claim 1, wherein the anti-static agent preferably contains an amidoamine to polyglycerol ester ratio of 50:50.

8. The polymeric material of claim 1, wherein the anti-static agent is the reaction produced between a C₈-C₁₈ saturated or unsaturated fatty acid and a Di C₁-C₈ amino C₁₋₈ amine.

9. The polymeric material of claim 1, wherein the polymer is a polyolefin.

10. The polymeric material of claim 1, wherein the polymer is a polypropylene.

11. The polymeric material of claim 1, wherein the polymer is a polyester, a polyamide, a styrene, a polylactic acid, or copolymers thereof.

12. The polymeric material of claim 1, wherein the anti-static agent is present in the polymer in an amount of from about 0.01% to 2% of total weight percent.

13. The polymeric material of claim 1, wherein the polymeric material has an initial surface resistance of about less than 10¹² ohms/square and wherein the polymeric material has a surface resistance of about less than 10¹⁰ ohms/square after 90 days.

14. An anti-static agent for a polymer composition comprising:
a polyglycerol ester combined with the following: 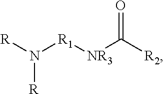
wherein R is CH₃(CH₂)ₓ, R₁ is (CH₂)_{y}, and R₂ is (CH₂)_{z}CH₃; and
wherein x is from 0 to 20, R₁ y is from 1 to 20, and z is from 0 to 20; and
wherein R₃ is H or a group that produces a quaternary compound.

15. The anti-static agent of claim 14, wherein the anti-static agent preferably contains an amidoamine to polyglycerol ester ratio of about 50:50.

16. The anti-static agent of claim 14, wherein the anti-static agent is a dimethylaminopropylamine.

17. The anti-static agent of claim 14, wherein the polyglycerol ester is chosen from the following formulas: 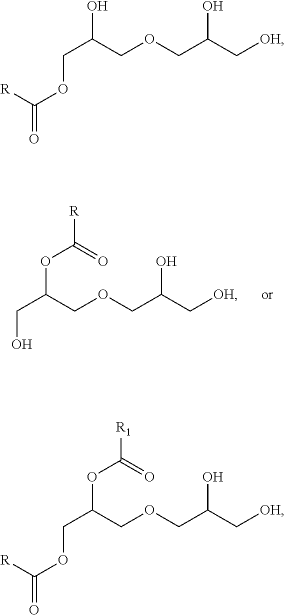
wherein R and R₁ are (CH₂)_{z}CH₃ and where z is from 0 to 16.

18. The anti-static agent of claim 16, wherein the polyglycerol ester is chosen from the following formulas: 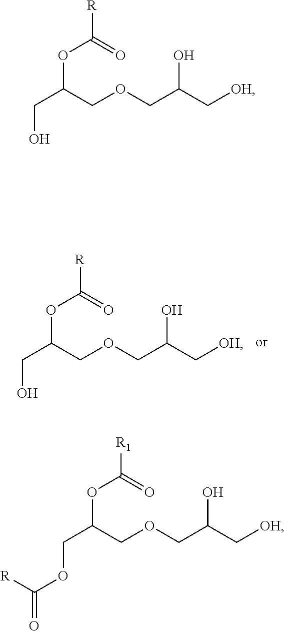
wherein R and R₁ are (CH₂)_{z}CH₃ and where z is from 0 to 16.

19. A polymer composition comprising:
a thermoplastic polymer;
an anti-static agent present in the polymer composition in an amount sufficient to reduce surface resistivity, the anti-static agent having the following formula: 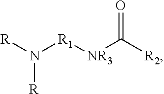
wherein R is CH₃(CH₂)ₓ, R₁ is (CH₂)_{y}, and R₂ is (CH₂)_{z}CH₃; and
wherein the anti-static agent is the reaction produced between a C₈-C₁₈ saturated or unsaturated fatty acid and a Di C₁-C₈ amino C₁-C₈ amine; and
wherein R₃ is H or a group that produces a quaternary compound.

20. The anti-static agent of claim 14, wherein the anti-static agent comprises a carboxylic acid with a dimethylaminopropylamine.
